(19) United States
(12) Patent Application Publication
(10) Pub. No.: US 2014/0037759 A1
(43) Pub. Date: Feb. 6, 2014

(54) Antacid Tablet

(71) Applicant: GlaxoSmithKline, LLC, Philadelphia, PA (US)

(72) Inventors: Maria Mirabile, Parsippany, NJ (US); Sandip G. Shah, Parsippany, NJ (US)

(73) Assignee: GlaxoSmithKline, LLC, Philadelphia, PA (US)

(21) Appl. No.: 13/755,229

(22) Filed: Jan. 31, 2013

Related U.S. Application Data
(60) Provisional application No. 61/594,055, filed on Feb. 2, 2012.

(51) Int. Cl. A61K 47/00

(52) U.S. Cl. CPC A61K 47/00
USPC 424/687

(57) ABSTRACT

Aspects of the present invention are directed to an oral antacid tablet comprising at least about 60% by weight directly compressible granulated calcium carbonate and an intense flavoring. The tablet may have a hardness of at least about 22 Strong-Cobb units and the tablet may have a mass of between about 500 mg and about 1,000 mg. Tablets of the present invention reduce or eliminate heartburn symptoms and also freshen breath.

### FIELD OF THE INVENTION

Aspects of the present invention are directed to antacid tablets, and, in particular, antacid tablets that freshen breath.

### BACKGROUND OF THE INVENTION

Calcium carbonate is a known antacid used to reduce or eliminate heartburn symptoms by neutralizing acid in an individual's stomach. Typical calcium carbonate tablets contain between 500 mg and 750 mg of calcium carbonate, although the size of the tablet is much greater. A typical calcium carbonate tablet weighs about 2000 mg, resulting in less than 50% of the tablet being the active material, i.e., calcium carbonate. The size of the tablet leads to the need for a large container to store the tablets and makes consumption of these tablets in public difficult. Often, individuals will forego consumption of an antacid in public and suffer through the discomfort of heartburn symptoms to avoid drawing attention to them by consuming a large antacid tablet.

In addition to discomfort, bad breath is often associated with heartburn symptoms. In fact, foods that are known to cause indigestion or heartburn are also known to cause bad breath. For example, fatty foods, fried foods, garlic, onions, and spicy foods can result in both stomach irritation and bad breath.

Sufferers of heartburn symptoms often find themselves taking breath mints or chewing gum to reduce bad breath. Breath mints or gum, however, do not reduce or eliminate excess stomach acid and do not reduce or eliminate stomach irritation.

Attempts to add mint flavors to antacid tablets have been made. These products, although effective at reducing heartburn symptoms suffer from some drawbacks. First, as mentioned before, these tablets are quite large and are not easily carried with a person in public. Second, although these tablets are flavored, they lack the intense flavor of traditional breath mints and thus provide little, if any, breath freshening. Additionally, these tablets often lack the hard crunchy texture commonly associated with breath mints

A single dosage form that can reduce or eliminate both heartburn symptoms and bad breath and that does not have the drawbacks associated with current dosage forms would be highly desirable.

### SUMMARY OF THE INVENTION

Aspects of the present invention are directed to an oral antacid tablet comprising at least about 60% by weight directly compressible granulated calcium carbonate and an intense flavoring. The tablet may have a hardness of at least about 22 Strong-Cobb Units and the tablet may have a mass of between about 500 mg and about 1,000 mg.

Tablets of the present invention may further include an antioxidant. Suitable antioxidants may include alpha tocopherol, beta tocopherol, gamma tocopherol, delta tocopherol, or combinations thereof.

In some embodiments, the tablet may comprise at least about 65% by weight directly compressible granulated calcium carbonate, or at least about 70% by weight directly compressible granulated calcium carbonate. In certain embodiments, the tablet may have a mass of between about 650 mg and about 850 mg or between about 700 mg and about 800 mg, and may comprise between about 400 mg and about 600 mg of calcium carbonate or between about 450 mg and about 550 mg of calcium carbonate.

Tablets of the present invention may also comprise between about 0.001% and about 10% by weight an intense flavoring or between about 0.001% and about 5% by weight an intense flavoring. The intense flavoring may contain any suitable flavor including, for example, peppermint, spearmint, wintergreen, cinnamon, a fruit flavor, or a combination thereof.

In certain embodiments, the hardness of the tablet may be at least about 25 Strong-Cobb Units or at least about 30 Strong-Cobb Units.

Tablets of the present invention may comprise directly compressible granulated calcium carbonate which granulation comprises at least about 75% by weight calcium carbonate, or at least about 85% by weight calcium carbonate, or at least about 95% by weight calcium carbonate.

Additional aspects of the present invention are directed to a method comprising reducing heartburn and freshening breath by ingesting a tablet of the present invention. In certain embodiments, the tablet is ingested after a meal.

### DETAILED DESCRIPTION OF THE INVENTION

Aspects of the present invention are directed to an oral antacid tablet comprising directly compressible granulated calcium carbonate and an intense flavoring. Oral antacid tablets of the present invention comprise both an antacid and breath freshener for the relief of heartburn symptoms and bad breath. The tablets alleviate many of the drawbacks associated with traditional methods to co-treat heartburn symptoms and bad breath. The tablets are smaller than traditional antacid tablets, making them more discrete and easier to consume in public. Additionally, the tablets of the present invention have several of the desirable characteristics of a breath mint For example, they have a strong breath freshening flavor and a hard texture, providing a similar mouth feel to a breath mint.

As used herein, the term “heartburn symptoms” includes heartburn related to indigestion, sour stomach, upset stomach, episodic and co-incidental heartburn with meals, and heartburn related to gastroesophageal reflux of acid stomach contents.

It is understood by those in the art that calcium carbonate has inherently poor compressibility, and, is not generally considered to be directly compressible. Calcium carbonate cannot simply be mixed with other excipients, binders and ingredients and put in a tablet press. Even if a tablet could be formed this way, it would have poor hardness and other mechanical properties. It was understood that to produce calcium carbonate tablets with proper characteristics, the calcium carbonate must first be granulated. Traditional calcium carbonate granulations contain less than about 50% by weight calcium carbonate, the rest of the granulation being filler and binders. These are not directly compressible because of the large amount of additional ingredients. Use of these granulations for calcium carbonate tablets resulted in very large tablets. Surprisingly, it was found that calcium carbonate granulations having a much higher amount of calcium carbonate and less fillers than traditional granulations are directly compressible and can be used in calcium carbonate tablets without the previously encountered mechanical property issues. In fact, tablets of the present invention produced using directly compressible calcium carbonate provide a desirable mouth feel, similar to that of a breath mint Directly compressible granulations of the present invention contain at least about 75% by weight calcium carbonate, or at least about 85% by weight calcium carbonate, or at least about 95% by weight calcium carbonate.

In certain embodiments, the calcium carbonate granulation may be made of a marbled source of calcium carbonate. The particle size of the calcium carbonate may be between about 2 and about 15 microns, preferably between about 4 and about 6 microns.

The granulation may be a wet granulation. In one embodiment, the granulation has the following particle size range: about 5% or less of the particles do not pass through a US#20 mesh; about 35% or greater of the particles do not pass through a US#60 mesh; and about 20% or less of the particles pass through a US#200 mesh.

The tablet of the present invention may contain at least about 60% by weight directly compressible granulated calcium carbonate, or at least about 65% by weight directly compressible granulated calcium carbonate, or at least about 70% by weight directly compressible calcium carbonate.

Using higher amounts of directly compressible calcium carbonate granulation that contain a higher amount of calcium carbonate results in a final dosage form that contains a higher percentage of calcium carbonate than traditional calcium carbonate tablets. For example, the final dosage form may contain at least about 50% by weight calcium carbonate, or, for example, at least about 65% by weight calcium carbonate, or, for example, at least about 80% by weight calcium carbonate. This allows for tablets that contain between about 400 mg and about 600 mg of calcium carbonate, or between about 450 mg and about 550 mg of calcium carbonate, or about 500 mg calcium carbonate, yet having a much smaller overall tablet size.

Tablets of the present invention provide for suitable stomach acid neutralization compared to larger, traditional calcium carbonate tablets. For example, tablets of the present invention may have an acid neutralization capacity (ANC) of greater than about 8.5 mEq per tablet, or greater than about 10 mEq per tablet, or greater than about 15 mEq per tablet.

A benefit of the current dosage form is that it has a smaller size than traditional antacid tablets. This smaller size allows users to consume tablets in a more discrete manner than traditional tablets. This results in increased use in social settings and improved treatment of heartburn symptoms. The tablets may have a mass of between about 500 mg and about 1,000 mg, or between about 650 mg and about 850 mg, or between about 700 mg and about 800 mg. In certain embodiments, the tablet has a mass of about 750 mg.

The tablets may have a variety of shapes, such as, for example, round, cylindrical, ring-shaped, star-shaped, among others. In a specific embodiment, the tablet is in the form of an oval-shaped cylinder having a major length of between about 0.7 inches and about 0.4 inches, a major width of between about 0.2 inches and about 0.4 inches, and a thickness of between about 0.2 inches and about 0.4 inches.

Another improvement of this invention over traditional calcium carbonate dosage forms is the hardness of the tablet. Traditional calcium carbonate tablets are soft to provide for ease of chewing or the ability to dissolve on the tongue. Tablets of the present invention are harder, providing for the mouth-feel of a breath mint The increased hardness provides for a more satisfying tactile experience of the user. In certain embodiments, the tablets have a hardness of at least about 22 Strong-Cobb Units (SCU), or at least about 25 SCU, or at least about 30 SCU.

Tablets of the present invention provide for the ability to not only treat heartburn symptoms, but to also freshen the breath of the user. Typically, foods associated with causing heartburn, i.e., onions, garlic, fatty foods, spicy foods, etc. are also associated with causing bad breath. A tablet that provides both heartburn relief and breath freshening would be highly desirable. Indeed, the flavored antacids in the prior art do not provide adequate breath freshening. Previously flavored calcium carbonate tablets were designed to simply mask the flavor of the calcium carbonate and various fillers to make consumption more palatable. They did not provide breath freshening.

Tablets of the present invention include an intense flavoring. These intense flavorings provide multiple benefits. First, they provide for a very strong flavor, allowing the tablet to provide fresh breath to the user. Additionally, the intensity of the flavor allows for a small amount of the flavorings to be used, consistent with the desire for a smaller tablet size. In certain embodiments, the intense flavorings have a total in-mouth impact of greater than about 5 flavor intensity units (fiu) on a traditional 15 point scale. In other embodiments, the intense flavoring may have a total in-mouth impact of greater than about 6 fiu on a traditional 15 point scale. In addition, the tablets of the present invention may continue to provide significant impact even after dissolving. For example, the tablets may have a total flavor intensity impact of greater than about 5 fiu 30 seconds after dissolving and greater than about 3.5 fiu 5 minutes after dissolving.

The intense flavorings may include among other things, a flavor and a cooling agent. Suitable cooling agents, include, for example, menthol and menthol derivatives. Suitable flavors include, for example, peppermint, spearmint, wintergreen, cinnamon, grapefruit, chocolate, cherry, raspberry, lemon, lime, strawberry, strawberry banana, orange, pineapple, passion fruit, mixed fruit, citrus berry, berry fusion, mixed berry, rainbow sherbet, or combinations thereof. The amount of intense flavoring present in the formulation may be from about 0.0015% to about 10% by weight of the composition or from about 0.1% to about 5.0% by weight of the composition.

An issue associated with flavored antacid tablets is the degradation of flavor intensity over time when exposed to elevated temperatures and humidity. This is of particular concern with antacids tablets because their stability is tested according to the International Conference on Harmonization (ICH) guidelines for stability testing of a drug product (40° C./75% RH). It was discovered that the addition of an antioxidant either directly to the intense flavoring or to the tablet formulation dramatically reduced flavor intensity degradation.

Suitable antioxidants for use in formulations of the present invention include, for example, alpha tocopherol, beta tocopherol, gamma tocopherol, delta tocopherol, butylated hydroxytoluene (BHT), butylated hydroxyanisole (BHA), ascorbic acid, fumaric acid, malic acid, ascorbyl palmitate, propyl gallate, sodium ascorbate, sodium metabisulfite, or combinations thereof. In certain embodiments, the antioxidant includes alpha tocopherol, beta tocopherol, gamma tocopherol, delta tocopherol, or a combination thereof. In some embodiments, the amount of antioxidant present in the formulation may be from about 0.01 to about 0.5% by weight of the composition or from about 0.02% to about 0.15% by weight of the composition.

Additionally, other conventional diluents or excipients may also be included, as needed, in the tablet. Suitable excipients which may be employed include, for example, disintegrants, fillers, binding agents, lubricants, compression aids, and wetting agents.

Tablets of the present invention may optionally contain suitable disintegrants such as, for example, sodium starch glycolate [Explotab®], crosslinked polyvinylpyrrolidone, corn starch, acacia, Croscarmellose of sodium [Ac-di-sol®], sodium carboxymethylcellulose, veegum, or alginates. The amount of disintegrant present may be from about 1% to about 10.0% by weight of the composition.

The tablets may also include additional diluents or fillers such as, for example, various grades of microcrystalline cellulose, such as Avicel PH1OI, Avicel PHI02, & Avicel PH200; corn starch; or Starch 1500. The amount of diluent or filler present in the formulation may be from about 1% to about 90.0% by weight of the composition. The dosage form may also optionally contain suitable lubricants or wetting agents, such as but not limited to, magnesium stearate, stearic acid and its pharmaceutically acceptable alkali metal salts, calcium stearate, sodium stearate, Cab-O-Sil, Syloid, sodium lauryl sulfate, sodium chloride, magnesium lauryl sulfate or talc. Preferably, a suitable lubricant is magnesium stearate or stearic acid. Preferably, a suitable wetting agent is a surfactant, such as sodium lauryl sulfate. The amount of lubricant present in the formulation may be from about 0.1% to about 10.0% by weight of the composition, whereas the amount of wetting agent may be from about 0.1-20% by weight.

The tablets may also include additional binding agents, such as, for example, polyvinylpyrrolidone, (PVP), or Providone 29K132. The amount of binding agent present in the formulation may be from about 0.1% to about 30.0% by weight of the composition. The tablet may also include coloring agents, or pigments, such as FD&C or D&C approved lakes and dyes, iron oxide and titanium dioxide. The amount of coloring agents or pigments present may be from about 0.1% to about 5.0% by weight of the composition.

In a certain embodiment, tablets of the present invention may comprise between about 65% and 75% by weight directly compressible granulated calcium carbonate, between about 20% and 30% by weight sorbitol powder, between about 1.5% and 3% by weight intense peppermint flavoring, between about 0.05% and 0.2% by weight sucralose, and between about 0.5% and 2% by weight calcium stearate. Tablets of the present invention are produced using standard tabletting processes known to those skilled in the art.

The tablets of the present invention may be consumed by humans upon the onset of heartburn symptoms or prior to the onset of heartburn symptoms as a preventive measure. The tablets may be macerated by the human until the entire tablet has been consumed or the human may allow the tablet to dissolve in his or her mouth.

The tablet may be consumed after a meal to provide relief from heartburn symptoms and/or freshen breath. For example, the tablet may be taken within 10 minutes after completion of the meal, or, for example, within 30 minutes after completion of the meal, or for example, within 60 minutes of the meal. It is, however, envisioned that this tablet may be consumed at any time during the day whenever the human desires relief from heartburn symptoms. The tablet may be ingested one at a time or multiple tablets. For example, 2, 3, or 4 tablets may be consumed at one time.

### EXAMPLES

### Example 1

### Breath Freshening Antacid Tablet

750 mg antacid tablets were formed with the following ingredients:

| Ingredient | % w/w |
|---|---|
| Directly Compressible Granulated Calcium Carbonate | 68% to 72% |
| (Nutri Granulations) | |
| Sorbitol Powder (NF) | 23% to 27% |
| Intense Peppermint Flavoring (including 0.04% to | 2% to 2.2% |
| 0.06% w/w of tablet Tocopherol) (Takasago Int. | |
| Corp) | |
| Sucralose (NF) | 0.06% to 0.1% |
| Calcium Stearate (NF) | 0.8 to 1.2% |
| Sugar Spheres (NF Blue 35-40 mesh size) | 1.8% to 2.2% |

A portion of the Intense Peppermint Flavoring with Tocopherol and Sucralose were mixed in a container to form a homogenous pre-blend.

A tote was then charged with Directly Compressible Granulated Calcium Carbonate (screened through 8 mesh screen or equivalent); the pre-blend of Intense Peppermint Flavoring with Tocopherol and Sucralose (screened through a 20 mesh screen); the remaining amount of the Intense Peppermint Flavoring with Tocopherol; Sugar Spheres (screened through a 8 mesh screen or equivalent); Sorbitol (screened through 8 mesh screen or equivalent); and Calcium Stearate (pre-screened through 20-mesh screen).

The mixture was blended to form a homogenous final blend. The final blend was compressed using a tablet press, the tooling comprising −0.3355″×0.5235″ oval shape punches and −0.3370″×0.5250″ dies, to form oval shaped tablets having a hardness of about 28 SCU.

### Example 2

### Pepperoni Pizza Study

A breath freshening study of the tablets of Example 1 was conducted to assess whether the tablets were suitable for combating bad breath associated with pepperoni pizza.

100 people were entered into and finished the study. The users reported to the study site without having performed oral hygiene in the previous 2 hours. The users were given two slices of pepperoni pizza to eat. They were then given the antacid tablets of the present invention to chew. Users rated their breath with respect to freshness upon arrival, 5 minutes after eating pepperoni pizza, immediately after taking the antacid tablets of the present invention and 5 minutes later.

A bi-polar scale from −15 (“most unfresh breath imaginable”) to +15 (“freshest breath imaginable”) was used. The only other point labeled was 0 (“neutral”). Upon arrival at the study site, the most common and median rating of breath freshness was a neutral rating of 0. Thirty people rated their breath as fresh; there were seven ratings of +10 or higher. Forty-seven people rated their breath as “unfresh;” there were ten ratings of −10 or lower.

Five minutes after eating pepperoni pizza, most people's breath was rated unfresh (rating <0). Eighty-eight ratings were unfresh with a median rating of −9. Only one person rated their breath as “neutral”.

Immediately after chewing two antacid tablets of the present invention, most people's breath became fresher. Ninety-six ratings were fresh (rating >0) with a median rating of +11. Sixty-two ratings were +10 or higher. Five minutes after chewing two antacid tablets of the present invention, most people's breath was still fresh. Ninety-three ratings were fresh with a median rating of +10. Fifty-nine ratings were +10 or higher.

Breath freshness from the initial assessment to the post-pizza rating indicated that breath became significantly more unfresh after eating pizza, p<0.0001. Eighty-eight people had ratings that decreased after eating pizza; the median change in ratings was a 7-unit decrease toward more unfresh breath.

Breath freshness from the post-pizza rating to the first post-consumption of antacid tablets of the present invention rating indicated that breath became significantly more fresh after chewing the antacid tablets of the present invention, p<0.0001. Ninety-five people had ratings that increased after chewing the antacid tablets of the present invention; the median change in ratings was a 19-unit increase toward fresher breath.

Breath freshness from the post-pizza rating to the second post-consuming antacid tablets of the present invention rating indicated that breath remained significantly more fresh 5 minutes after chewing the antacid tablets of the present invention, p<0.0001. Ninety-seven people had ratings that that were still fresher 5-minutes after chewing the antacid tablets of the present invention; the median change in ratings was an 18.5-unit change toward more fresh breath.

What is claimed is:

1. An oral antacid tablet comprising:
At least about 60% by weight directly compressible granulated calcium carbonate; and
An intense flavoring;
wherein the tablet has a hardness of at least about 22 Strong-Cobb Units; and
wherein the tablet has a mass of between about 500 mg and about 1,000 mg.

2. The tablet of claim 1, further comprising an antioxidant.

3. The tablet of claim 2, wherein the antioxidant is alpha tocopherol, beta tocopherol, gamma tocopherol, delta tocopherol, or a combination thereof.

4. The tablet of claim 1, wherein there is at least about 70% by weight directly compressible granulated calcium carbonate.

5. The tablet of claim 1, wherein the mass is between about 700 mg and about 800 mg.

6. The tablet of claim 1, further comprising between about 400 mg and about 600 mg of calcium carbonate.

7. The tablet of claim 1, wherein the intense flavoring is present in an amount of between about 0.001% and about 5% by weight.

8. The tablet of claim 1, wherein the intense flavoring has a flavor of peppermint, spearmint, wintergreen, cinnamon, a fruit flavor, or a combination thereof.

9. The tablet of claim 1, wherein the hardness is at least about 25 Strong-Cobb Units.

10. The tablet of claim 1, wherein the directly compressible granulated calcium carbonate comprises at least about 75% by weight calcium carbonate.

11. A method comprising reducing heartburn and freshening breath by ingesting a tablet of claim 1.

12. The method of claim 11, wherein ingestion of the tablet of claim 1 occurs after a meal.
